(19) United States
(12) Patent Application Publication
(10) Pub. No.: US 2010/0305356 A1
(43) Pub. Date: Dec. 2, 2010

(54) METHOD FOR PRODUCING ISOCYANATES

(75) Inventors: Gerhard Olbert, Dossenheim (DE); Torsten Mattke, Freinsheim (DE); Carsten Knoesche, Niederkirchen (DE); Andreas Daiss, Deidesheim (DE); Jens Denecke, Speyer (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/675,137

(22) PCT Filed: Aug. 13, 2008
(86) PCT No.: PCT/EP2008/060636

§ 371 Date: Feb. 25, 2010

(30) Foreign Application Priority Data
Aug. 30, 2007 (EP) 07115342.3

(51) Int. Cl. C07C 263/10; B01F 15/02; B01J 19/24

(52) U.S. Cl. 560/347; 366/177.1; 422/224

(57) ABSTRACT

The present invention relates to a process for preparing isocyanates.

To prepare isocyanates by phosgenating the corresponding amines, there is in principle the possibility of a liquid phase phosgenation or of a gas phase phosgenation. In the gas phase phosgenation, the reaction conditions are selected such that at least the diamine, diisocyanate and phosgene reaction components, but preferably all reactants, products and reaction intermediates, are gaseous under these conditions, more preferably until the reaction is complete. The present invention relates especially to gas phase phosgenation.

EP 1 275 639 A1 describes the gas phase phosgenation of (cyclo)aliphatic diamines in a reaction zone with constrictions of the walls.

In the mixing device, the amine- and phosgene-containing reactant streams are fed coaxially to a mixing zone, the phosgene-containing reactant stream being conducted in the interior and the amine-containing reactant stream in the exterior. In the region in which the reactant streams are combined, i.e. the reaction zone, there is a further reduction or slight enlargement of the flow cross section, such that the flow rate rises owing to the volume increase in the course of the reaction.

A disadvantage of this arrangement is that the amine stream is conducted coaxially in the exterior. This can result in solid formation on the walls of the mixing device, since the amine is present in excess compared to the phosgene at the walls, which promotes by-product formation. A further disadvantage of the process is that, in the case of excessive acceleration of the flow, the cross-sectional constriction can result in dampening of the turbulent variable speeds in the flow, which are crucial for the rapid mixing in a turbulent flow.

It is likewise stated in EP 1275639A1 that swirling of the reactant streams should be effected in the mixing apparatus before the reactant streams are combined such that the turbulent variable speeds in the reactant flows are increased and the mixing is then effected more rapidly when the two reactant streams are combined.

EP 1526129 A1 describes the increase in the vortexing in a mixing nozzle by swirl-generating internals. This generates tangential vortexing of the overall stream, which does not, however, have a significant effect on the mixing of the different streams with one another.

EP 1 275 640 A1 describes the gas phase phosgenation of (cyclo)aliphatic di- and triamines in a mixing tube with a reactor, in which the gas flow in the mixing region is accelerated.

A disadvantage of this process is that the maximum speed difference between the reactant streams is not achieved immediately at the start of mixing, and hence the minimum possible mixing time is not achieved either.

DE 10359627 A1 discloses a gas phase phosgenation in which amine is mixed in by means of a concentric annular gap between two phosgene streams, where the areas through which the phosgene streams flow are in a ratio of from 1:0.5 to 1:4.

International application WO 2007/028715 discloses a process in which amine and phosgene are metered in through an annular gap, i.e. a ring-shaped continuous gap.

In all of these documents, exclusively smooth nozzles are disclosed, which may comprise swirl-generating internals if appropriate.

The flow of the reactants into the mixing chamber is usually turbulent in the mixing units disclosed. The flow profile has a turbulent core flow and a wall interface layer. The wall interface layer consists of a laminar underlayer close to the wall and a laminar-turbulent transition area. In the interface layer, especially the laminar underlayer, lower speeds of flow exist than in the core. At the contact point of the reactant feeds, an area of low speed and consequently high residence time is thus formed. There may be formation and deposition of solids there.

The slower interface layer, on entry into the mixing chamber, additionally reduces the shear rate between jet and environment and hence the edge turbulence which causes mixing (starting of the free jet). Consequently, the mixing time is increased. A reduction in the interface layer accordingly leads to a reduced deposition tendency and a shorter mixing time.

It was thus an object of the present invention to develop a reaction regime for a gas phase phosgenation, with which industrial scale performance becomes possible and which brings about a reduction in the interface layer thickness at the opening point of the reactant streams into the mixing chamber. It is another object of the invention to develop a mixing nozzle which has a very high turbulence intensity in the core of the reactant streams at the opening point, such that very rapid mixing of the reactant streams over the entire nozzle cross section should proceed.

The object is achieved by processes for preparing isocyanates by reacting the corresponding amines with phosgene, optionally in the presence of at least one, preferably exactly one, inert medium, in the gas phase, by contacting fluid streams of amine and phosgene and subsequently reacting them with one another, which comprises reducing the turbulent flow interface of at least one stream immediately before it is contacted with the other stream by means of at least one fluidic flow disruptor.

This measure simultaneously increases the level of turbulence in the core of the reactant streams.

The present invention further provides an apparatus for mixing at least two different fluid substances, comprising at least one flow channel per fluid substance, in which at least one of the flow channels, upstream of the point at which the different substances come into contact with one another for the first time, has at least one flow disruptor.

The present invention further provides for the use of such apparatus in chemical reactions, in which fluid chemical compounds are mixed with one another.

It is known, for example, from EP 289840 B1 or from EP 1275639 A1 to mix amine and phosgene in the gas phase phosgenation with the aid of a combination of nozzle and annular gap. This mixing principle is shown as an illustration in FIG. 1.

The disruption to the flow is, preferably in accordance with the invention, generated by those fluidic flow disruptors 4 or 5 which, in the flow channel in question, i.e. before the mixing of the components, generate displacement of the flow by virtue of a widening (FIGS. 2 and 3) or a constriction (FIGS. 4 and 5) of limited length.

The action of the flow disruptors 4 or 5 in the flow channel is such that they force a displacement of the flow. Beyond the flow disruptor and a recirculation area which forms, the flow aligns itself to the wall again and the turbulent interface layer forms again. In this starting phase, the interface layer thickness is reduced compared to the flow conditions upsteam of the flow disruptor. The opening point should be very close to the alignment point in order to realize a minimum interface layer thickness. The opening point must not, however, be upstream of the alignment point of the flow to the wall, since there is otherwise recirculation from the mixing chamber into the reactant feed.

To describe the invention, the following parameters (see FIGS. 2a and 4a) are employed:

The diameter D is the diameter or the gap width of the particular flow channels, measured in each case at the site of combination of the streams to be mixed, i.e. the point at which the streams to be mixed can have the first possible contact.

In the case of a constriction of the flow channel, the height of the flow disruptor 5 is described by the parameter d1, in the case of a widening by means of a flow disruptor 4 by the parameter d2.

The length of the flow disruptor is described by the parameter e, the distance of the flow disruptor upstream of the site of combination of the streams to be mixed by the parameter L (see figures).

The height d1 or depth d2 of the flow disruptors 5 and 4 and their length e must, in accordance with the invention, be sufficient to generate a displacement and the formation of a recirculation area in fluidic terms.

The distance L must be greater than the length of the recirculation area which forms. However, it should be significantly smaller than the starting zone for complete formulation of a turbulent flow.

This depends on the type and speed of the flowing fluid and can be determined by the person skilled in the art experimentally or by simulations.

The mechanical design of such flow disruptors must generate a displacement of the flow in fluidic terms and the formation of a recirculation area, but it is not important in accordance with the invention in what manner the flow disruptors are designed.

Cross sections of illustrative designs of flow disruptors are shown in FIG. 6:

a: rectangles
b: trapeziums
c: rhombuses in flow direction (arrow)
d: rhombuses against flow direction (arrow)
e: semi- or part-circles
f: sawteeth in flow direction (arrow)
g: sawteeth against flow direction (arrow)
h: polygons
i: triangles.

Preference is given to a, b, e, h and i, particular preference to a, b, e and i, very particular preference to a and e and special preference to a.

The d1:D ratio is preferably from 0.002 to 0.2:1, more preferably from 0.05 to 0.18:1, even more preferably from 0.07 to 0.15:1 and especially from 0.1 to 0.12:1.

The distance L is preferably greater than twice the height d1, more preferably greater than four times and most preferably eight times the parameter d1. The length L is preferably less than fifty times the diameter D, more preferably less than twenty times and most preferably less than ten times the diameter D.

In the case of a widening, the distance L is preferably greater than the depth d2, more preferably greater than twice and most preferably six times the depth d2. The length L is preferably less than fifty times the diameter D, more preferably less than twenty times and most preferably less than ten times the diameter D.

In the case of a depression, d2:D is from 0.001 to 0.5:1, more preferably from 0.01 to 0.3:1 and most preferably from 0.1 to 0.2:1.

In the case of a constriction, the d1:l ratio is of minor importance and is generally from 10:1 to 1:10, preferably from 5:1 to 1:5 and more preferably from 2:1 to 1:2.

In the case of a widening, the d2:l ratio should generally be from 2:1 to 1:20, preferably from 1:1 to 1:15 and more preferably from 1:2 to 1:10.

Whether a constriction or a widening of the flow cross section is preferable depends upon whether an increased turbulence level is desired in the core of the reactant flow. A noticeable increase in the turbulence level arises only through a constriction of the cross section. In contrast, a widening, compared to a constriction, of the cross section brings about a more efficient reduction in the thickness of the laminar interface layer.

In contrast to EP 1526129 A1, the flow disruptors are, in accordance with the invention, mounted on the walls of the flow channels, i.e. the diameter D is constricted by d1 from “the outside inward”, while the oblique plates and helical elements disclosed in EP 1526129 A1 are mounted as turbulence generators in the interior of the flow channel and thus constrict the diameter D “from the inside outward”.

The only embodiment disclosed explicitly in the example in EP 1526129 A1 fills the flow channel completely.

The mixing device disclosed in EP 1 275 639 A1 discloses a constriction only in the region in which mixing has already set in or taken place. This promotes the risk of formation of deposits or blockages. In contrast, the subject matter of the present invention is to generate a displacement and recirculation before the mixing.

In addition, the flow disruptors may enclose an angle (phi) with the flow direction (FIG. 7, plan view).

An angle φ=0° means that the flow disruptor is transverse to the flow direction (arrow); φ=90° means that the flow disruptor is aligned to the flow direction (in flow direction). Preferably, φ is from 0 to 80°, more preferably from 0 to 60°, even more preferably from 0 to 45°, in particular from 0 to 30°, and φ is especially 0°.

By virtue of angles φ≠0, a tangential speed vector (swirl) is generated in the particular flow, in addition to the inventive axial turbulence.

It has been found that streams in which the inventive flow disruptors generate a displacement and a recirculation area upstream of the opening mix better with one another. In the case of mixing of phosgene and amine as streams, this leads to formation of a lower level of deposits in the region in which the streams are contacted with one another than when the mixing is effected without fluidic flow disruptors.

The thickness of the laminar interface layer in fully developed turbulent flow is, according to Prandtl, (62.7×D)/(Re^{0.875}) in which Re is the Reynolds number of the fluid under the existing conditions. According to W. Bohl, “Technische Strömungslehre” [Technical Flow Theory], 12th edition, Vogel-Buchverlag, 2001, this gives, for the area proportion a of the laminar underlayer at the opening cross section, a=1−(1-2×62.7/(Re^{0.875})²). According to this, for a Reynolds number of 5000, the laminar interface layer takes up approx. 14% of the opening cross section. In this 14% of the cross-sectional area, accordingly, there exists a laminar flow with low speeds. When the improvement principle described is implemented in an optimal manner, this laminar region can be prevented almost completely. Accordingly, the zones of slow flow rate close to the wall can be prevented and hence also the formation of deposits. Furthermore, the jet now enters the mixing zone with high peripheral speed, such that enhanced peripheral turbulence and therefore better mixing are achieved.

The mixing device may preferably be a static mixing unit, for example a nozzle mixing device, for example coaxial mixing nozzles, Y or T mixers, jet mixers or mixing tubes.

In a coaxial jet mixer, one component (preferably the amine) is conducted into the other component (which is then preferably phosgene) at high speed through a concentric tube with a small diameter (nozzle) in a mixing tube.

The reactors may, for example, be cylindrical reaction spaces without internals and without moving parts.

One embodiment of a mixing/reaction unit is described in EP 1275639 A1, and there particularly in paragraphs [0013] to [0021] and the example together with FIG. 1, which is hereby incorporated in the present disclosure by reference. Preference is given, however, in contrast to the disclosure there, to the metered addition of the amine through the internal tube and of phosgene as the outer stream.

One embodiment of a mixing/reaction unit is described in EP 1275640 A1, and there particularly in paragraphs [0010] to [0018] and the example together with FIG. 1, which is hereby incorporated in the present disclosure by reference. Preference is given, however, in contrast to the disclosure there, to the metered addition of the amine through the internal tube and of phosgene as the outer stream.

A further embodiment of a mixing/reaction unit is described in EP 1319655 A2, and there particularly in paragraphs [0015] to [0018] and the example together with FIG. 1, which is hereby incorporated into the present disclosure by reference.

It may be advisable to install flow homogenizers, as described in EP 1362847 A2, and there particularly in paragraphs [0008] to [0026] and the example together with FIG. 1, which is hereby incorporated in the present disclosure by reference.

Also conceivable is the use of a plurality of nozzles aligned in parallel, as described in EP 1449826 A1, and there particularly in paragraphs [0011] to [0027] and Example 2 together with FIGS. 1 to 3, which is hereby incorporated in the present disclosure by reference.

A further embodiment of a mixing/reaction unit is described in DE 10359627 A1, and there particularly in paragraphs [0007] to [0025] and Example 1 together with the figure, which is hereby incorporated in the presence disclosure by reference.

A preferred embodiment of a mixing nozzle is a slot mixing nozzle, as described in WO 2008/55898, and there particularly from page 3 line 26 to page 15 line 31, and a reaction chamber as described there from page 15 line 35 to page 31 line 38, together with the figures, which is hereby incorporated in the present disclosure by reference.

A particularly preferred embodiment of a mixing nozzle is an annular gap mixing nozzle, as described in international patent application WO 2007/028715, and there particularly from page 2 line 23 to page 11 line 22, and a reaction chamber as described there from page 11 line 26 to page 21 line 15 together with FIG. 2, which is hereby incorporated in the present disclosure by reference.

It is essential to the invention that a flow disruptor is installed in the course of at least one of the streams to be mixed in the nozzle.

To prevent solids deposition and blockages, the phosgene-containing reactant stream is preferably conducted in the inventive mixing device such that all apparatus walls, after the reactant streams have been combined, are flowed over by the phosgene-containing reactant stream(s) and the amine-containing reactant stream(s) is/are enclosed completely by the phosgene-containing reactant stream(s) until complete mixing of the streams or substantially complete conversion of the amine has been effected.

Preference is therefore given to metering in the amine in the interior, such that the stream is surrounded completely on all sides by a phosgene stream.

The amines which can be used in a gas phase phosgenation have to satisfy particular requirements (see below).

The amines may be monoamines, diamines, triamines or higher-functionality amines, preferably diamines. This accordingly gives rise to the corresponding monoisocyanates, diisocyanates, triisocyanates or higher-functionality isocyanates, preferably diisocyanates.

The amines and isocyanates may be aliphatic, cycloaliphatic or aromatic, preferably aliphatic or cycloaliphatic and more preferably aliphatic.

Cycloaliphatic isocyanates are those which comprise at least one cycloaliphatic ring system.

Aliphatic isocyanates are those which have exclusively isocyanate groups which are bonded to straight or branched chains.

Aromatic isocyanates are those which have at least one isocyanate group bonded to at least one aromatic ring system.

In the context of this application, (cyclo)aliphatic isocyanates are an abbreviated representation of cycloaliphatic and/or aliphatic isocyanates.

Examples of aromatic diisocyanates are preferably those having 6-20 carbon atoms, for example monomeric methylene 2,4′- or 4,4′-di(phenyl isocyanate) (MDI), tolylene 2,4- and/or 2,6-diisocyanate (TDI) and naphthyl 1,5- or 1,8-diisocyanate (NDI).

Diisocyanates are preferably (cyclo)aliphatic diisocyanates, more preferably (cyclo)aliphatic diisocyanates having from 4 to 20 carbon atoms.

Examples of customary diisocyanates are aliphatic diisocyanates such as tetramethylene 1,4-diisocyanate, pentamethylene 1,5-diisocyanate, hexamethylene diisocyanate (1,6-diisocyanatohexane), octamethylene 1,8-diisocyanate, decamethylene 1,10-diisocyanate, dodecamethylene 1,12-diisocyanate, tetradecamethylene 1,14-diisocyanate, derivatives of lysine diisocyanate, tetramethylxylylene diisocyanate (TMXDI), trimethylhexane diisocyanate or tetramethylhexane diisocyanate, and also 3 (or 4), 8 (or 9)-bis(isocyanatomethyl)tricyclo[5.2.1.0^{2,6}]decane isomer mixtures, and also cycloaliphatic diisocyanates such as 1,4-, 1,3- or 1,2-diisocyanatocyclohexane, 4,4′- or 2,4′-di(isocyanatocyclohexyl)methane, 1-isocyanato-3,3,5-trimethyl-5-(isocyanatomethyl)cyclohexane (isophorone diisocyanate), 1,3- or 1,4-bis(isocyanatomethyl)cyclohexane, 2,4- or 2,6-diisocyanato-1-methylcyclohexane.

Preference is given to pentamethylene 1,5-diisocyanate, 1,6-diisocyanatohexane, 1-isocyanato-3,3,5-trimethyl-5-(isocyanatomethyl)cyclohexane, 4,4′-di(isocyanatocyclohexyl)methane and tolylene diisocyanate isomer mixtures. Particular preference is given to 1,6-diisocyanatohexane, 1-isocyanato-3,3,5-trimethyl-5-(isocyanatomethyl)cyclohexane and 4,4′-di(isocyanatocyclohexyl)methane.

For the process according to the invention, it is possible to use those amines for the reaction to give the corresponding isocyanates for which the amine, its corresponding intermediates and the corresponding isocyanates are present in gaseous form under the selected reaction conditions. Preference is given to amines which, during the reaction, decompose under the reaction conditions to an extent of at most 2 mol %, more preferably to an extent of at most 1 mol % and most preferably to an extent of at most 0.5 mol %. Particularly suitable here are amines, especially diamines, based on aliphatic or cycloaliphatic hydrocarbons having from 2 to 18 carbon atoms. Examples thereof are 1,5-diaminopentane, 1,6-diaminohexane, 1-amino-3,3,5-trimethyl-5-aminomethylcyclohexane (IPDA) and 4,4′-diaminodicyclohexylmethane. Preference is given to using 1,6-diaminohexane (HDA).

It is likewise possible to use aromatic amines for the process according to the invention, which can be converted to the gas phase without significant decomposition. Examples of preferred aromatic amines are tolylenediamine (TDA), as the 2,4- or 2,6-isomer or as a mixture thereof, for example as an 80:20 to 65:35 (mol/mol) mixture, diaminobenzene, 2,6-xylidine, napthyldiamine (NDA) and 2,4′- or 4,4′-methylene(diphenylamine) (MDA) or isomer mixtures thereof. Among these, preference is given to the diamines, particular preference to 2,4- and/or 2,6-TDA.

In the gas phase phosgenation, the aim is by definition that the compounds which occur in the course of the reaction, i.e. reactants (diamine and phosgene), intermediates (especially the mono- and dicarbamyl chlorides which form as intermediates), end products (diisocyanate), and any inert compounds metered in, remain in the gas phase under the reaction conditions. Should these or other components be deposited out of the gas phase, for example on the reactor wall or other apparatus components, these depositions can undesirably change the heat transfer or the flow of the components in question. This is especially true of amine hydrochlorides which occur, which form from free amino groups and hydrogen chloride (HCl), since the resulting amine hydrochlorides precipitate out readily and are only reevaporable with difficulty.

The reactants, or else only one of them, may be metered into the mixing chamber together with at least one inert medium.

The inert medium is a medium which is present in the reaction chamber in gaseous form at the reaction temperature and does not react with the compounds which occur in the course of the reaction. The inert medium is generally mixed with amine and/or phosgene before the reaction, but may also be metered in separately from the reactant streams. For example, nitrogen, noble gases such as helium or argon, or aromatics such as chlorobenzene, chlorotoluene, o-dichlorobenzene, toluene, xylene, chloronaphthalene, decahydronaphthalene, carbon dioxide or carbon monoxide may be used. Preference is given to using nitrogen and/or chlorobenzene as the inert medium.

In general, the inert medium is used in an amount such that the ratio of the gas volumes of inert medium to amine or to phosgene is from more than 0.0001 to 30, preferably from more than 0.01 to 15, more preferably from more than 0.1 to 5.

Before the process according to the invention is carried out, the starting amines are evaporated and heated to from 200° C. to 600° C., preferably from 300° C. to 500° C., and fed to the reactor through the mixing device, if appropriate diluted with an inert gas or with the vapors of an inert solvent.

Before performing the process according to the invention, the phosgene used in the phosgenation is likewise heated to a temperature within the range from 200° C. to 600° C., preferably from 300° C. to 500° C., if appropriate diluted with an inert gas or with the vapors of an inert solvent.

In a preferred embodiment, the amine streams are heated to a temperature up to 50° C. higher than the phosgene streams, preferably to a temperature up to 30° C., more preferably up to 24° C. and most preferably up to 20° C. higher. The temperature of the amine streams is preferably at least 5° C., more preferably at least 10° C., above that of the phosgene streams.

According to the invention, phosgene is used in excess based on amino groups. Typically, a molar ratio of phosgene to amino groups of from 1.1:1 to 20:1, preferably from 1.2:1 to 5:1 is present.

The mixing and reaction of the two gaseous reactants takes place in the process according to the invention after the introduction of the diamine and phosgene reactant streams, via the slots as entry areas, in the mixing chamber as the reaction chamber.

The reaction generally sets in with contact of the reactants immediately after the mixing.

Thus, the mixing of the reactants, if appropriate mixed with inert medium, takes place in the front part of the reaction chamber (mixing chamber).

To perform the inventive reaction, the preheated stream comprising amine or mixtures of amines and the preheated stream comprising phosgene are passed continuously into the reactor, preferably a tubular reactor.

The reactors consist generally of steel, glass, alloyed or enameled steel, and have a length which is sufficient to enable full reaction of the diamine with the phosgene under the process conditions.

It may be advisable to incorporate flow homogenizers into the reactant lines, as known, for example, from EP 1362847A. For homogenization of the speed of the reactant streams, however, preference is given to a long initial length in the reactant line relative to the diameter of the feed line, which is from 2 to 40 times the feed line diameter, more preferably from 4 to 30 times, most preferably from 5 to 20 times.

A constriction of the flow cross section as described in patent EP 1275640A1 after the combination of the reactant streams to prevent backflows is possible, but preference may be given to dispensing with one.

Following the concept of the invention, in order that an amine stream, after the start of mixing, has no contact with the apparatus walls but rather is surrounded by phosgene-containing reactant streams, the amine stream 1 is metered in between phosgene streams 2.

In accordance with the invention, this has the effect that the uppermost and the lowermost or the outermost is in each case a phosgene stream which keeps the amine stream(s) away from the walls of the reactor.

The flow cross sections of the phosgene-containing reactant stream(s) is/are configured such that the characteristic mixing length measure again becomes as small as possible. Since the phosgene reactant is supplied in stoichiometric excess and, moreover, the phosgene speed is preferably less than the amine speed, a greater cross-sectional area has to be selected than for the amine-containing stream, which also gives rise to greater characteristic dimensions. The mixing path length is selected at less than 200 mm, preferably less than 100 mm, more preferably less than 50 mm, even more preferably less than 25 mm and especially less than 10 mm. The mixing path length is defined as the maximum distance that the fluid elements of two or more reactant streams have to pass through at right angles to the flow direction of the reactant streams until molecular mixing of the reactant streams has been effected.

The ratio of the total area of the amine streams to the total area of the phosgene streams is greater than 0.00002, preferably greater than 0.0002, more preferably greater than 0.002 and most preferably greater than 0.02.

The ratio of the total area of the amine streams to the total area of the phosgene streams is less than 5, preferably less than 1, more preferably less than 0.5 and most preferably less than 0.2.

The area ratio of two phosgene-conducting areas separated by an amine-conducting slot is from 0.1 to 10, preferably from 0.2 to 5, more preferably from 0.4 to 2.5, very particularly from 0.8 to 1.25, in particular from 0.9 to 1.1 and especially 1.

Since the intensity and rapidity of the mixing of the amine- and phosgene-containing reactant streams depend significantly on the shear gradient to be established in the mixing zone, the mixing zone has to be configured such that the shear gradient is particularly high.

To this end, the speed difference between the amine- and phosgene-containing reactant streams should firstly be selected at a particularly high level and the characteristic length dimensions should secondly be selected at a minimum level, since the shear gradient is proportional to the quotient of speed difference and characteristic length dimension.

Since the speed difference between the amine- and phosgene-containing reactant streams should be high, either the phosgene-containing or the amine-containing reactant streams must have a high speed. Since the amine feeds to the mixing zone are relatively sensitive to the formation of deposits and blockages and backflow in the amine feed should be avoided in any case, the flow rate of the amine-containing reactant stream is preferably selected to be greater than the speed of the phosgene-containing reactant stream.

The higher the speed of the amine-containing reactant streams, the higher the speed of the phosgene-containing reactant streams can also be selected at the same shear rate. A higher phosgene speed brings about smaller flow cross sections of the phosgene feed and hence smaller mixing path lengths and hence more rapid mixing. In order to achieve a very high amine speed, the aim is therefore to establish a local Mach number of greater than 0.6 in the amine stream at the point of combination with the phosgene stream.

The Mach number means the ratio between local flow rate and local speed of sound. In a particular embodiment of the process, feed rate of the amine-containing reactant streams is selected such that exactly a Mach number of 1 is present at the exit of the amine streams into the mixing zone.

In the case of a so-called adjusted amine feed rate, the pressure of the amine stream at this point corresponds exactly to the pressure of the phosgene-containing reactant stream at the point of combination. In the case of an unadjusted amine feed rate, the pressure of the amine stream at the exit from the amine feed is greater than the pressure of the phosgene-containing stream at the combination. In this case, there is then further expansion of the amine-containing stream, which is associated with a pressure drop down to the pressure of the phosgene-containing stream. Whether a nozzle is operated adjusted or unadjusted depends on the upstream pressure of the amine-containing stream and of the phosgene-containing stream upstream of the mixing nozzle.

In a further particular embodiment, the amine feed rate is configured such that Mach numbers of greater than 1 are achieved actually in the feeds. This can be achieved, for example, by configuring the feed of the amine-containing streams in the form of one or more Laval nozzles, which feature initial narrowing of the flow cross section until a Mach number of one is attained and then widening again, which leads to a further expansion and acceleration of the flow. In order to achieve supersonic flow (Mach number greater than 1), the ratio of the amine tank pressure to the mixing zone pressure must be greater than the so-called critical pressure ratio. The higher the pressure ratio and the higher the tank temperature of the amine stream, the higher is the maximum achievable speed.

Since the amine reactant is often damaged thermally at excessively high temperatures, however, no excessively high temperatures can be established. The upstream amine pressure also cannot be increased as desired owing to the amine vapor pressure. Preference is therefore given to configuring the amine feed such that, in the amine-containing reactant stream, directly at the combination with the phosgene-containing stream, or, in the case of an unadjusted nozzle, just downstream thereof, Mach numbers of from 0.6 to 4, more preferably from 0.7 to 3, even more preferably from 0.8 to 2.5 and especially from 0.9 to 2.0 are established.

The Mach numbers specified can be converted by the person skilled in the art to flow rates in a simple manner with a known tank temperature and known substance data. Equally, the person skilled in the art can calculate the upstream pressure required depending on the given Mach number and the substance data.

The high entry speed of the amine stream into the mixing zone serves, as stated above, to achieve a very large speed difference between amine-containing and phosgene-containing reactant streams. Moreover, the high flow rate locally reduces the system pressure and hence also the reactant concentrations and the temperature, which leads to a reduction in the reaction rates and hence to a simplification of the mixing task.

In order to achieve very short mixing path lengths, the aim must be likewise to select the flow rate of the phosgene-containing reactant stream at as high as possible a level, but without too greatly reducing the speed difference between amine-containing and phosgene-containing reactant stream. To this end, the cross-sectional area of the phosgene stream is selected so as to give rise to a Mach number of from 0.2 to 2.0, preferably from 0.3 to 1.5, more preferably from 0.4 to 1.0, even more preferably from 0.5 to 1.0 and especially from 0.7 to 1.0.

The flow cross sections of the amine-containing reactant streams are configured in the inventive mixing unit such that, firstly, a high operational stability is ensured and, otherwise, very short mixing path lengths are maintained. Therefore, length dimensions characteristic for the supply of the amine-containing reactant streams of from 0.5 to 50 mm, preferably from 0.75 to 25 mm, more preferably from 1 mm to 10 mm and most preferably from 1 mm to 5 mm are selected. The characteristic length dimension means the smallest length measure of the flow cross section, i.e., in the case of a gap, the gap width, or, in the case of a circular orifice, the orifice diameter.

The individual reactants in the mixing device are preferably conducted into the reactor with a flow rate of from 20 to 400 meters/second, preferably from 25 to 300 meters/second, more preferably from 30 to 250 meters/second, even more preferably from 50 to 200 meters/second, in particular from more than 150 to 200 meters/second and especially from 160 to 180 meters/second.

In one possible embodiment of the invention, it may be advisable to introduce the phosgene streams, especially the outer phosgene stream, into the mixing chamber with a higher flow rate than the amine stream that they surround, more preferably at least 10 m/s more, even more preferably at least 20 m/s more and especially at least 50 m/s more.

However, it may also be possible and advisable to introduce the outer phosgene stream into the mixing chamber with a higher flow rate than the amine stream, and the inner phosgene stream with a lower flow rate. This constitutes a further possible embodiment of the present invention.

In a preferred embodiment of the invention, it is advisable to introduce the phosgene streams, especially the outer phosgene stream, into the mixing chamber with a lower flow rate than the amine stream that they surround, more preferably at least 50 m/s less, even more preferably at least 60 m/s less, even more preferably 80 m/s less and especially at least 100 m/s less.

In a preferred embodiment of the present invention, in the case of a multitude of phosgene streams, these are connected to exactly one phosgene feed line with a low pressure drop and without additional regulating devices, such that the rate with which the phosgene flows is about the same.

Equally, in the case of a multitude of amine streams, they are preferably connected to exactly one amine line with a low pressure drop without additional regulating devices, such that the speed with which the amine flows is about the same.

However, it is also possible to connect the phosgene and/or amine streams in the slots to one separately regulated feed line each, such that the speeds are adjustable individually and independently of one another for each line.

The reactants enter the mixing chamber with a speed vector. The speed vector can be resolved into an axial, radial and tangential direction component. The axial direction is understood to mean the direction component of the speed vector parallel to the longitudinal axis of the mixing space. The radial direction is understood to mean the direction component of the speed vector from outside toward the longitudinal axis, i.e. enclosing a right angle with the longitudinal axis. Tangential direction is understood to mean the direction component of the speed vector parallel to the edge of the mixing chamber, i.e. a circular peripheral motion.

For the mixing of the reactant streams, an improvement in the mixing which is established can be achieved by the incorporation of elements which generate a tangential speed, for example into the feed line of the substreams of the excess components into the mixing chamber. A suitable tangential speed-generating element would, for example, be a spiral-twisted belt (helix) introduced into the feed line, round or rectangular guide plates (guide paddles) or the like. The action of the tangential speed-generating internals is to increase the shear between flow layers of different composition in the flow of the nozzle.

To generate a tangential speed, tangential entry of the feed line of one or more reactant streams is also possible, or, in the case of radial inflow of one or more reactant streams, a ring of paddles.

In addition, it may be advisable to introduce the phosgene and amine streams into the mixing chamber with contra rotatory tangential speed, for example by metering the phosgene streams into the mixing chamber with a clockwise tangential speed viewed along the longitudinal axis of the reactor, and the intervening amine stream with an anticlockwise tangential speed.

The angle enclosed by the cumulative vector formed from the vectors of the tangential speed and from the vector of the axial speed of the streams thus metered in enclosed with the longitudinal axis of the reactor may be from 5 to 85°, preferably from 17 to 73°, more preferably from 30 to 60° for one set of streams, for example the phosgene streams, and from −5 to −85°, preferably from −17 to −73°, more preferably from −30 to −60° for the other streams, for example the amine stream.

In addition, it is advisable to meter the flows into the mixing chamber with different radial speeds. In this case, an angle is established between the cumulative vector formed from the radial speed vector and from the axial speed vector with the longitudinal axis. This angle corresponds generally to the angle of the corresponding metering channel with the longitudinal axis of the mixing chamber. A negative angle means metered addition from the inside outward, a positive angle metered addition from the outside inward; an angle of 0° means a flow parallel to the longitudinal axis of the mixing chamber and an angle of 90° a flow at right angles to the longitudinal axis of the mixing chamber.

The outer phosgene stream can be metered into the mixing chamber through the mixing device at a radial angle of from 0 to 85°, preferably from 5 to 85°, more preferably from 7 to 65°, even more preferably from 15 to 35° and especially from 18 to 30°.

The amine stream can be metered into the mixing chamber through the mixing device at a radial angle of from −50° to +50°, preferably from −25 to 25°, more preferably from −10 to 10° and most preferably from −3 to +3°.

The inner phosgene stream can be metered into the mixing chamber through the mixing device at a radial angle of from 0 to −85°, preferably from −5 to −85°, more preferably from −7 to −65°, even more preferably from −15 to −35° and especially from −18 to −30°.

It is advantageous when the outer phosgene stream and amine stream, relative to one another, enclose a radial angle of from 1 to 60°, preferably from 7 to 50°, more preferably from 15 to 45° and more preferably from 18 to 35°.

It is also advantageous when the amine stream and inner phosgene stream, relative to one another, enclose a radial angle of from 1 to 60°, preferably from 10 to 50°, more preferably from 15 to 45° and more preferably from 18 to 35°.

In order to achieve substantially complete conversion of the amine to the particular product of value, a mixing time of the phosgene-containing stream with the amine-containing stream of less than 10 ms, preferably less than 5 ms, more preferably less than 2 ms, even more preferably less than 1 ms and especially less than 0.5 ms is achieved by the measures described above. The mixing time is defined as the maximum time needed by the fluid elements which exit from the amine feed until a phosgene/amine ratio of greater than or equal to 4 is established therein. The time is counted in each case from the exit of a fluid element out of the amine feed.

### Reaction Chamber

The reaction chamber comprises, in the front region, the mixing chamber in which the mixing of the gaseous mixture of phosgene, amine, if appropriate mixed with inert medium, predominantly takes place, which is generally accompanied by the onset of the reaction. In the rear part of the reaction chamber, essentially only the reaction then takes place and, to a minor degree at most, the mixing.

For the purposes of distinction, the mixing chamber can refer to the region of the reaction chamber in which the mixing of the reactants takes place to a degree of 99%. In a preferred embodiment of the present invention, the conversion in the mixing chamber, i.e. the consumption of the amine used, is less than 15%. The degree of mixing is specified as the ratio of the difference of the locally averaged mixing ratio and of the starting mixing ratio before mixing relative to the difference of the mean final mixing ratio after mixing and of the initial mixing ratio before mixing. Regarding the concept of the mixing ratio, see, for example, J. Warnatz, U. Maas, R. W. Dibble: Verbrennung [Combustion], Springer Verlag, Berlin Heidelberg, N.Y., 1997, 2nd edition, p. 134.

Reactor is understood to mean the technical apparatus which comprises the reaction chamber. It may be all customary reaction chambers known from the prior art which are suitable for the noncatalytic, monophasic gas reaction, preferably for the continuous noncatalytic, monophasic gas reaction, and which withstand the moderate pressures required. Suitable materials for the contact with the reaction mixture are, for example, metals such as steel, tantalum, nickel, nickel alloys, silver or copper, glass, ceramic, enamel or homogeneous or heterogeneous mixtures thereof. Preference is given to using steel reactors. The walls of the reactor may be hydraulically smooth or profiled. Suitable profiles are, for example, cracks or waves.

It may be advantageous when the material used, preferably the material used for the mixing device and/or the reactor and more preferably that used for the reactor, has a low roughness, as described in unpublished International patent application PCT/EP2007/063070 with the filing date Nov. 30, 2007, which is hereby incorporated fully in the context of the present disclosure by reference.

It is generally possible to use the reactor designs known from the prior art. Examples of reactors are known from EP-B1 289840, column 3 line 49—column 4 line 25, EP-B1 593334, WO 2004/026813, page 3 line 24—page 6, line 10, WO 03/045900, page 3 line 34—page 6 line 15, EP-A1 1275639, column 4 line 17—column 5 line 17, and EP-B1 570799, column 2 line 1—column 3 line 42, each of which is incorporated explicitly in the scope of this disclosure by reference.

Preference is given to using tubular reactors.

It is likewise possible to use essentially cuboidal reaction chambers, preferably plate reactors or plate reaction chambers. A particularly preferred plate reactor has a ratio of width to height of at least 2:1, preferably at least 3:1, more preferably at least 5:1 and especially at least 10:1. The upper limit in the ratio of width to height depends upon the desired capacity of the reaction chamber and is in principle not limited. Technically viable reaction chambers have been found to be those with a ratio of width to height up to 5000:1, preferably up to 1000:1.

The reaction of phosgene with amine in the reaction chamber is effected at absolute pressures of from more than 0.1 bar to less than 20 bar, preferably between 0.5 bar and 15 bar and more preferably between 0.7 and 10 bar. In the case of reaction of (cyclo)aliphatic amines, the absolute pressure is most preferably between 0.7 bar and 5 bar, in particular from 0.8 to 3 bar and especially from 1 to 2 bar.

In general, the pressure in the feed lines to the mixing apparatus is higher than the above-specified pressure in the reactor. According to the selection of the mixing apparatus, at this pressure declines. The pressure in the feed lines is preferably higher by from 20 to 2000 mbar, more preferably from 30 to 1000 mbar, than in the reaction chamber.

In one possible embodiment, the reactor consists of a bundle of reactors. In one possible embodiment, the mixing unit need not be an independent apparatus; instead, it may be advantageous to integrate the mixing unit into the reactor. One example of an integrated unit composed of mixing unit and reactor is that of a tubular reactor with flanged-on nozzles.

In the process according to the invention, the reaction of phosgene with amine is effected in the gas phase. Reaction in the gas phase is understood to mean that the conversion of the reactant streams and intermediates to the products react with one another in the gaseous state and, in the course of the reaction during passage through the reaction chamber, remain in the gas phase to an extent of at least 95%, preferably to an extent of at least 98%, more preferably to an extent of at least 99%, even more preferably to an extent of at least 99.5%, in particular to an extent of at least 99.8% and especially to an extent of at least 99.9%.

Intermediates are, for example, the monoamino monocarbamoyl chlorides, dicarbamoyl chlorides, monoamino monoisocyanates and monoisocyanato monocarbamoyl chlorides formed from the diamines, and also the hydrochlorides of the amino compounds.

In the process according to the invention, the temperature in the reaction chamber is selected such that it is above the boiling point of the diamine used, based on the pressure conditions existing in the reaction chamber. According to the amine used and pressure established, an advantageous temperature in the reaction chamber of more than 200° C., preferably more than 260° C. and more preferably more than 300° C. typically arises. In general, the temperature is up to 600° C., preferably up to 570° C.

The mean contact time of the reaction mixture in the process according to the invention is generally between 0.001 second and less than 5 seconds, preferably from more than 0.01 second to less than 3 seconds, more preferably from more than 0.015 second to less than 2 seconds. In the case of reaction of (cyclo)aliphatic amines, the mean contact time may even more preferably be from 0.015 to 1.5 seconds, in particular from 0.015 to 0.5 second, especially from 0.020 to 0.1 second and often from 0.025 to 0.05 second.

Mean contact time is understood to mean the time lapse from the beginning of mixing of the reactants until they leave the reaction chamber into the workup stage. In a preferred embodiment, the flow in the reactor of the process according to the invention is characterized by a Bodenstein number of more than 10, preferably more than 100 and more preferably of more than 500.

In a preferred embodiment, the dimensions of the reaction chamber and the flow rates are selected such that a turbulent flow is present for the reaction mixture, i.e. a flow with a Reynolds number of at least 2300, preferably at least 2700, the Reynolds number being formed with the hydraulic diameter of the reaction chamber.

The gaseous reaction mixture preferably flows through the reaction chamber with a flow rate of from 10 to 300 meters/second, preferably from 25 to 250 meters/second, more preferably from 40 to 230 meters/second, even more preferably from 50 to 200 meters/second, in particular from more than 150 to 190 meters/second and especially from 160 to 180 meters/second.

As a result of the turbulent flow, narrow residence time distributions with a low standard deviation of usually not more than 6%, as described in EP 570799, and good mixing are achieved. Measures, for example the constriction described in EP-A-593 334, which is additionally prone to blockage, are not necessary.

It may be advisable to incorporate flow homogenizers into the reactor, as known, for example, from EP 1362847A.

The reaction volume may be temperature-controlled over its outer surface. In order to build production plants with a high plant capacity, it is possible to connect a plurality of reactor tubes in parallel. However, the reaction can also preferably be effected adiabatically. This means that heating or cooling energy streams do not flow over the outer surface of the reaction volume by technical measures.

In a preferred embodiment, the reaction conditions are selected such that the reaction gas at the exit from the reaction chamber has a phosgene concentration of more than 25 mol/m³, preferably from 30 to 50 mol/m³. Moreover, at the exit from the reaction chamber, an inert medium concentration of more than 25 mol/m³, preferably of from 30 to 100 mol/m³, is generally present.

The reaction chamber may have a uniform diameter or have a series of constrictions or widenings in the course of the flow. This is described, for example, in WO 2007/028715, page 14 line 29 to page 20 line 42, which is hereby explicitly incorporated into the present disclosure.

However, the configuration of the reaction chamber, in accordance with the invention, does not play any role in the mixing of the components.

The volume of the reactor which is flowed through can be filled with static mixers, for example packings, shaped bodies, fabrics, perforated or slotted sheets; however, the volume is preferably very substantially free of internals.

The installation of guide plates into the reaction chamber is also conceivable. A suitable turbulence-generating element would, for example, be an inserted spiral-twisted belt, round or angular oblique plates or the like.

In order to maintain short mixing path lengths and hence short mixing times even in the case of large amine and phosgene flow rates, as are customary in isocyanate production on the industrial scale, one possibility is parallel connection of many small mixing nozzles with an adjoining mixing and reaction zone, in which case the parallel-connected units are separated from one another by walls. The advantage of this process variant lies in a relatively favorable length to diameter ratio of the mixing and reaction zones. The larger this ratio is, the more favorable (narrower) is the residence time distribution of the flow. At the same residence time and flow rate, it is thus possible by virtue of many parallel-connected units that the length to diameter ratio is increased and hence the residence time distribution is also narrowed. In order to minimize the apparatus complexity, the individual reaction zones open into a combined postreaction zone, in which the remaining conversion of the amine is effected.

### Quench

After the reaction, the gaseous reaction mixture is washed with a solvent, preferably at temperatures greater than 130° C. (quench). Preferred solvents are hydrocarbons which are optionally substituted by halogen atoms, for example hexane, benzene, nitrobenzene, anisole, chlorobenzene, chlorotoluene, o-dichlorobenzene, trichlorobenzene, diethyl isophthalate (DEIP), tetrahydrofuran (THF), dimethylformamide (DMF), xylene, chloronaphthalene, decahydronaphthalene and toluene. The solvent used is more preferably monochlorobenzene. The solvent used may also be the isocyanate. In the wash, the isocyanate is transferred selectively to the wash solution. Subsequently, the remaining gas and the resulting wash solution are separated, preferably by means of rectification, into isocyanate, solvent, phosgene and hydrogen chloride.

Once the reaction mixture has been converted in the reaction chamber, it is conducted into the workup apparatus with quench. This is preferably a so-called wash tower, wherein the isocyanate formed is removed from the gaseous mixture by condensation in an inert solvent, while excess phosgene, hydrogen chloride and, if appropriate, the inert medium pass through the workup apparatus in gaseous form. Preference is given to keeping the temperature of the inert solvent above the dissolution temperature of the carbamoyl chloride corresponding to the amine in the selected quench medium. Particular preference is given to keeping the temperature of the inert solvent above the melting point of the carbamoyl chloride corresponding to the amine.

In general, the pressure in the workup apparatus is lower than in the reaction chamber. The pressure is preferably lower by from 50 to 500 mbar, more preferably from 80 to 150 mbar, than in the reaction chamber.

The wash can, for example, be carried out in a stirred vessel or in other conventional apparatus, for example in a column or mixer-settler apparatus.

In process technology terms, it is possible to use all extraction and washing processes and apparatus known per se for a wash in the process according to the invention, for example those which are described in Ullmann's Encyclopedia of Industrial Chemistry, 6th ed, 1999 Electronic Release, chapter: Liquid—Liquid Extraction—Apparatus. For example, these may be one-stage or multistage, preferably one-stage, extractions, and also those in cocurrent or countercurrent mode, preferably countercurrent mode.

The quench may, for example, be designed as described in EP 1403248 A1, and there particularly in paragraphs [0006] to [0019] and the example together with FIGS. 1 and 2, which is hereby incorporated in the present disclosure by reference.

The quench may, for example, be designed as described in WO2008/055904, and there particularly from page 3 line 30 to page 11 line 37, together with Example 1 and the figures, which is hereby incorporated in the present disclosure by reference.

The quench may, for example, be designed as described in WO2008/055904, and there particularly from page 3 line 26 to page 16 line 36, together with Example 1 and the figures, which is hereby incorporated in the present disclosure by reference.

The quench may preferably be designed as described in WO 2005/123665, and there particularly from page 3 line 10 to page 8 line 2 and the example, which is hereby incorporated in the present disclosure by reference.

In this quench zone, the reaction mixture, which consists essentially of the isocyanates, phosgene and hydrogen chloride, is mixed intensively with the liquid sprayed in. The mixing is effected by lowering the temperature of the reaction mixture proceeding from 200 to 570° C. to from 100 to 200° C., preferably to from 140 to 180° C., and transferring the isocyanate present in the reaction mixture completely or partly by condensation into the liquid droplets sprayed in, while the phosgene and the hydrogen chloride remain essentially completely in the gas phase.

The proportion of the isocyanate present in the gaseous reaction mixture which is transferred into the liquid phase in the quench zone is preferably from 20 to 100% by weight, more preferably from 50 to 99.5% by weight and especially from 70 to 99% by weight, based on the isocyanate present in the reaction mixture.

The reaction mixture preferably flows through the quench zone from the top downward. Below the quench zone is arranged a collecting vessel in which the liquid phase is separated out, collected, removed from the reaction chamber via an outlet and then worked up. The remaining gas phase is removed from the reaction chamber via a second outlet and likewise worked up.

The quench can be effected, for example, as described in EP 1403248 A1 or as described in international application WO 2005/123665.

To this end, the liquid droplets are generated by means of one-substance or two-substance atomizer nozzles, preferably one-substance atomizer nozzles, and, according to the embodiment, generate a spray cone angle of from 10 to 140°, preferably from 10 to 120°, more preferably from 10° to 100°.

The liquid which is sprayed in via the atomizer nozzles must have a good solubility for isocyanates. Preference is given to using organic solvents. In particular, aromatic solvents which may be substituted by halogen atoms are used.

In a particular embodiment of the process, the liquid sprayed in is a mixture of isocyanates, a mixture of isocyanates and solvent, or isocyanate, in which case the quench liquid used in each case may have proportions of low boilers, such as HCl and phosgene. Preference is given to using the isocyanate which is prepared in the particular process. Since the lowering of the temperature in the quench zone causes the reaction to stop, side reactions with the isocyanates sprayed in can be ruled out. The advantage of this embodiment is especially that removal of the solvent can be dispensed with.

In an alternative preferred embodiment, the inert medium which is used together with at least one of the reactants and the solvent which is used in the quench are the same compound; in this case, very particular preference is given to using monochlorobenzene.

Small amounts of by-products which remain in the isocyanate can be separated from the desired isocyanate by means of additional rectification, by stripping with an inert gas or else crystallization, preferably by rectification.

In the subsequent optional purification stage, the isocyanate is removed from the solvent, preferably by distillation. It is likewise possible here to remove residual impurities, comprising hydrogen chloride, inert medium and/or phosgene, as described, for example, in DE-A1 10260092.

The present invention further provides a mixing device comprising at least one flow channel 1 around which are arranged, on both sides, at least two flow channels 2 such that the orifices of the flow channels 1 and 2 open in a mixing chamber, and at least one of the flow channels 1 and 2 with a diameter D has at least one flow disruptor of height d1 and/or at least one flow disruptor of depth d2 at a distance L from the opening into the mixing chamber, where the d₁:D ratio is from 0.002 to 0.2:1, more preferably from 0.05 to 0.18:1, even more preferably from 0.07 to 0.15:1 and especially from 0.1 to 0.12:1, or the ratio d₂:D is from 0.001 to 0.5:1, more preferably from 0.01 to 0.3:1 and even more preferably from 0.1 to 0.2:1, and the distance L in the case of an increase is greater than twice the height d1, more preferably greater than 4 times and even more preferably 8 times the size d1. The length L is preferably less than 50 times the diameter D, more preferably less than 20 times and most preferably less than 10 times the diameter D. In the case of a depression, the distance L is preferably greater than the depth d2, more preferably greater than twice and most preferably six times the depth d2. The length L is preferably less than fifty times the diameter D, more preferably less than twenty times and most preferably less than ten times the diameter D.

The data given in the text above apply to this inventive apparatus.

The action of this inventive apparatus is based on the generation of displacement of the flow or of formation of a recirculation area with subsequent reformation of the turbulent interface layer. The smaller interface layer brought about as a result in the buildup phase brings about higher shear rates between jet and environment beyond the opening and hence shorter mixing times.

This inventive principle can be applied generally to procedures in which rapid mixing of fluid, i.e. gaseous or liquid, substances is desired, especially in chemical reactions.

Such chemical reactions are preferably those in which solid substances are formed as end products or intermediates under the reaction conditions. The cause of the solids formation is local oversaturation of the solid-forming component with respect to the equilibrium solubility. The more rapid the mixing, the higher the oversaturation is too. A high oversaturation leads to the formation of more solid nuclei and generally to smaller primary particles. When this is an intermediate, small primary particles react further more rapidly than large primary particles, since they have more surface area. The rate of the subsequent reaction thus depends crucially on the size of the particles formed. For high space-time yields, very small particles therefore have to be generated in the mixing unit. Moreover, the formation of relatively large particles leads to the risk of formation of deposits in the mixing unit. To prevent solid deposits and to achieve short mixing times, small interface layers are therefore the aim.

This principle is applicable both to monophasic and polyphasic, mutually miscible or immiscible media.

Advantageously, the inventive apparatus can be used in the preparation of isocyanates by reacting the corresponding amines with phosgene, as a mixing apparatus for the mixing of amine and phosgene. It is at first unimportant whether the reaction takes place in the gas phase or in the liquid phase; it may particularly advantageously be used as a mixing apparatus in the gas phase phosgenation.

A further advantageous reaction in which the inventive apparatus is employed as a mixing apparatus is the preparation of diaminodiarylmethanes by condensing the corresponding amines with formaldehyde or its storage compounds. These storage compounds are, for example, commercial aqueous formalin solutions, paraformaldehyde, trioxane or highly concentrated formalin solutions. Instead of or in a mixture with formaldehyde, it is also possible to use at least one compound which releases formaldehyde. In particular, the formaldehyde is used as an aqueous formalin solution, alcoholic formalin solution, hemiacetal, methyleneimine of a primary amine, or N,N′-methylenediamine of a primary or secondary amine, and also paraformaldehyde.

Particular mention should be made of the preparation of 2,4′- and 4,4′-methylenediphenylamine (MDA) isomer mixtures from formaldehyde and aniline. In general, this reaction is acid-catalyzed.

Such processes are common knowledge and are described, for example, in Kunststoffhandbuch [Polymer Handbook], volume 7, Polyurethane [Polyurethanes], Carl Hanser Verlag Munich Vienna, 3rd edition, 1993, pages 76 to 86, and in a large number of patent applications, for example DE 100 31 540 or WO 99/40059. By virtue of the variation of the ratio of acid to aniline and of formaldehyde to aniline, the proportion of the 2-ring product in the crude MDA can be adjusted as desired.

For its preparation, the reactants are metered continuously into a reactor in the desired ratio relative to one another, and an amount of reaction product equal to the feed stream is withdrawn from this reactor. The reactors used are, for example, tubular reactors. In the continuous or semicontinuous mode, the reactants are metered into a batch reactor preferably provided with a stirrer and/or a pumped circulation system, from which the fully reacted reaction product is withdrawn and sent to workup.

Preference is given to performing the preparation preferably at a molar ratio of aniline to formaldehyde greater than 2. The molar ratio of acid (as catalyst) to aniline is preferably greater than 0.05. Under these conditions, there is increased formation of the particular two-ring product in the reaction mixture.

The continuous reaction is preferably performed at a temperature in the range between 0 and 200° C., preferably between 20 and 150° C. and especially between 40 and 120° C. It has been found that the proportion of the 2,2′- and 2,4′-isomers in the reaction product rises with the increase in the temperature.

The pressure in the reaction is from 0.1 to 50 bar absolute, preferably from 1 to 10 bar absolute.

In the batchwise and semicontinuous performance of the reaction, on completion of metered addition of the feedstocks, the reaction mixture can be subjected to a so-called aging. To this end, the reaction mixture is left in the reactor or transferred to another, preferably stirred, reactor. The temperature of the reaction mixture is preferably above 75° C., especially within a range between 110 and 150° C.

The preparation of the condensation product is followed by a workup, which is not relevant for the use of the inventive mixing nozzles in the process.

The advantage of the use of the inventive mixing nozzles in the preparation of diaminodiarylmethanes is that more rapid mixing and finer dispersion of droplets in the polyphasic reaction mixture is realized. In this way, formaldehyde can be reacted rapidly to give the desired intermediate. Areas with a high formaldehyde concentration, which leads to the formation of N-methylated by-products (N-methyl-MDA), can be reduced, such that a lower level of by-product is formed.

### 1. FIGURES

FIG. 1: Mixing of amine and phosgene in gas phase phosgenation with the aid of a combination of nozzle and annular gap

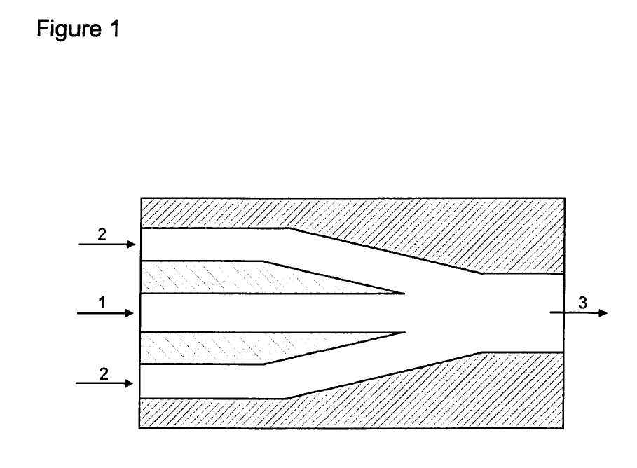

FIG. 2: Embodiment of the present invention with widening of the channel

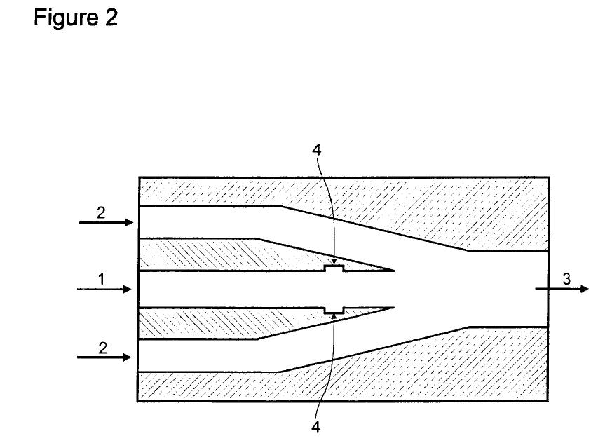

FIG. 2a: Definition of the parameters D, L, d2

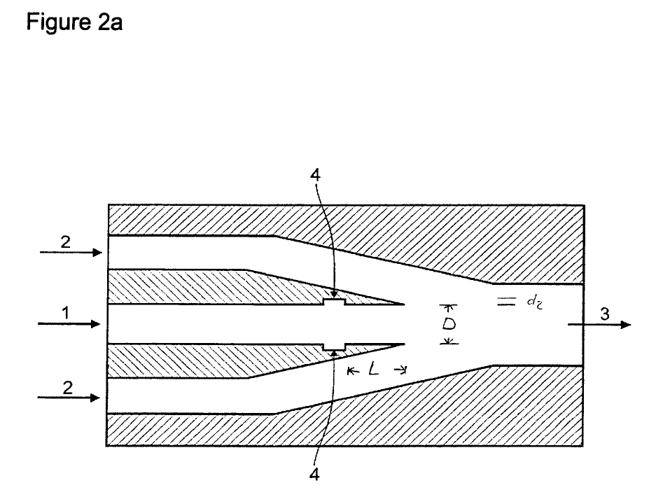

FIG. 3: Embodiment of the present invention with widening of the channel

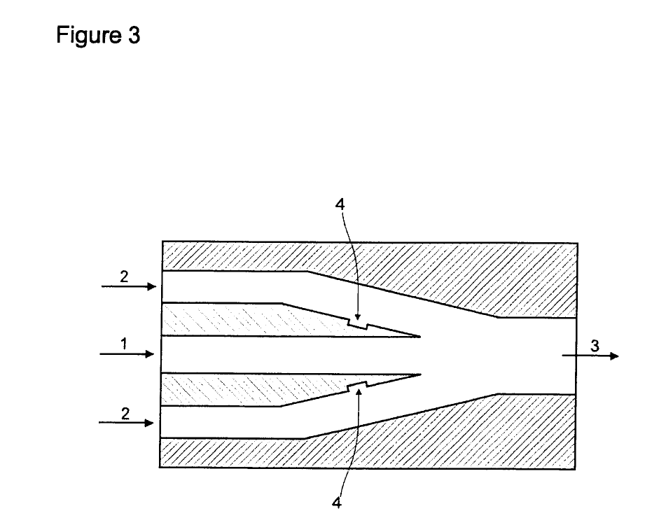

FIG. 4: Embodiment of the present invention with constriction of the channel

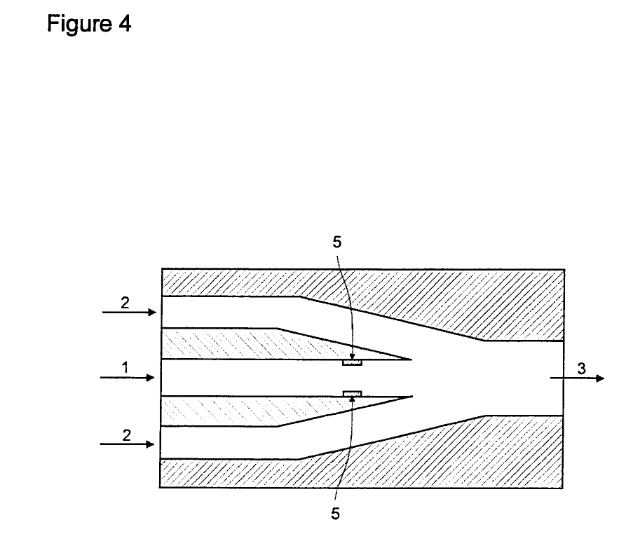

FIG. 4a: Definition of the parameters D, L, d1

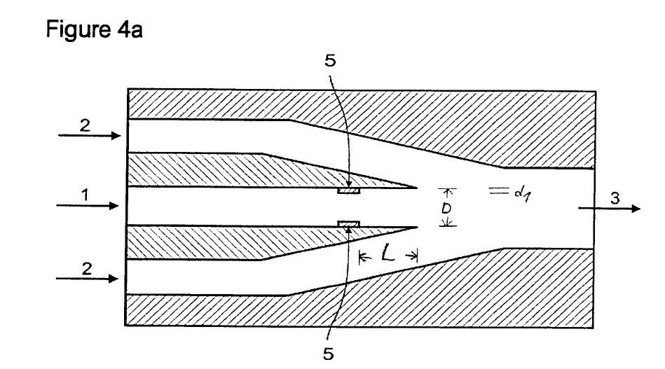

FIG. 5: Embodiment of the present invention with constriction of the channel

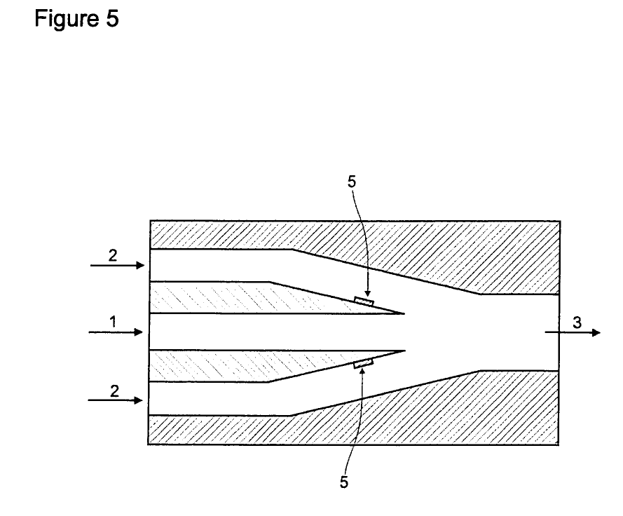

FIG. 6: Illustrative embodiments of flow disruptors

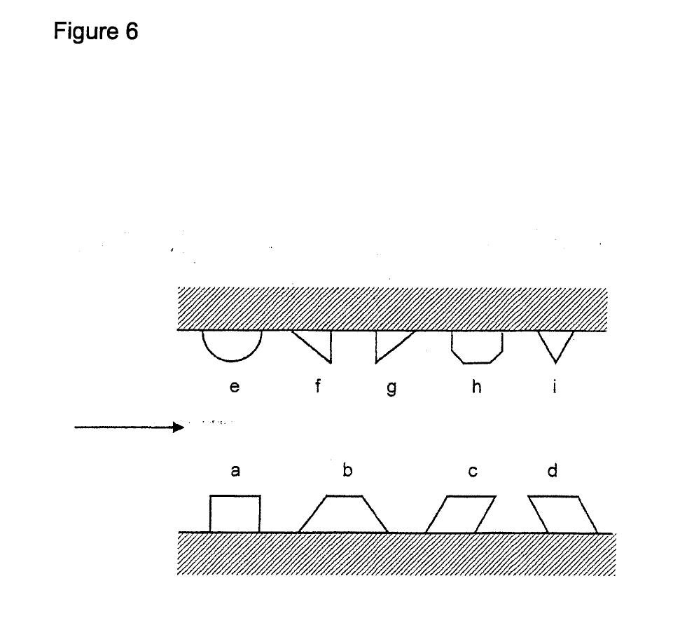

FIG. 7: Definition of the angle φ (phi) of flow disruptors

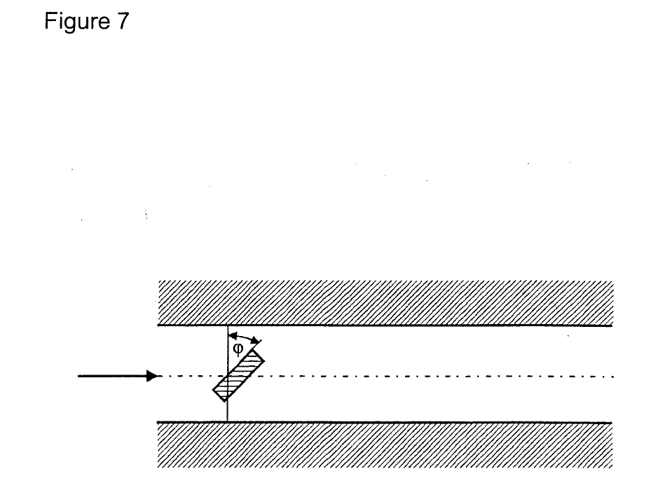

### LIST OF REFERENCE NUMERALS IN THE FIGURES

1 Amine stream
2 Phosgene stream
3 Reaction mixture
4 Flow disruptor
5 Flow disruptor

1. A process for preparing an isocyanate by reacting in the gas phase the corresponding amine with phosgene, optionally in the presence of at least one inert medium, by contacting fluid streams of amine and phosgene and subsequently reacting them with one another, which comprises reducing the turbulent flow interface of at least one stream immediately before it is contacted with the other stream by means of at least one fluidic flow disruptor.

2. The process according to claim 1, wherein the fluidic flow disruptor consists in a widening of limited length in a flow channel, through which the fluid stream in question is conducted.

3. The process according to claim 2, wherein the ratio of the depth d2 of the widening to the diameter D of the flow channel is from 0.001 to 0.5:1.

4. The process according to claim 1, wherein the fluidic flow disruptor consists in a constriction of limited length in a flow channel through which the fluid stream in question is conducted.

5. The process according to claim 4, wherein the ratio of the height d1 of the constriction to the diameter D of the flow channel is from 0.002 to 0.2:1.

6. The process according to claim 1, wherein the flow disruptors have a shape selected from the group consisting of rectangles, trapeziums, rhombuses, semicircles, part-circles, sawteeth, polygons and triangles.

7. The process according to claim 1, wherein the flow disruptors enclose an angle Φ (phi) with the flow direction, where Φ may be from 0 to 80°.

8. The process according to claim 1, wherein the mixing is effected in a mixing apparatus selected from the group consisting of coaxial mixing nozzles, Y mixers, T mixers, jet mixers and mixing tubes.

9. The process according to claim 1, wherein the mixing is effected by means of annular gap mixing nozzles or slot nozzles.

10. A mixing device comprising at least two flow channels which are arranged such that the openings of the flow channels open in a mixing space, at least one of the flow channels at a diameter D having at least one flow disruptor of height d₁ and/or at least one flow disruptor of depth d₂ at a distance L from the opening into the mixing chamber, where the ratio of d₁:D is from 0.002 to 0.2:1 or the ratio d₂:D is from 0.001 to 0.5:1 and the distance L is at least twice the diameter D.

11. An apparatus for mixing fluid substances comprising the mixing device according to claim 10.

12. An apparatus for mixing fluid substances in chemical reactions, in which solid substances are formed as end products or intermediates under the reaction conditions comprising the mixing device according to claim 10.

13. An apparatus for the preparation of an isocyanate by reacting the corresponding amine with phosgene by mixing comprising the mixing device according to claim 10.

14. An apparatus for the preparation of diaminodiarylmethanes by condensing the corresponding amines with formaldehyde or its storage compounds comprising the mixing device according to claim 10.
